(19) United States
(12) Patent Application Publication
(10) Pub. No.: US 2016/0338637 A1
(43) Pub. Date: Nov. 24, 2016

(54) SLEEP MONITORING DEVICE HOST COMPUTER AND SLEEP MONITORING DEVICE

(71) Applicant: SHENZHEN MEDICA TECHNOLOGY DEVELOPMENT CO., LTD, Shenzhen (CN)

(72) Inventors: Jinpeng SHEN, Shenzhen (CN); Jinfeng HUANG, Shenzhen (CN); Peng DAI, Shenzhen (CN)

(21) Appl. No.: 15/117,176

(22) PCT Filed: Oct. 31, 2014
(86) PCT No.: PCT/CN2014/090049

§ 371 Date: Aug. 5, 2016

(30) Foreign Application Priority Data
May 30, 2014 (CN) 201420287342.1

(51) Int. Cl. A61B 5/00

(52) U.S. Cl. CPC A61B 5/4806; A61B 5/6887

(57) ABSTRACT

The present disclosure provides a host for sleep monitoring device which includes a host body and a switch arranged on the host body. The host for sleep monitoring device further includes a top cover and a second magnet arranged on the inner surface of the top cover. The host body includes a housing and a first magnet arranged on the outer surface of the housing. The host body and the top cover are pulled together by the first magnet and the second magnet. The present disclosure further provides a sleep monitoring device. In the embodiments of the present disclosure, since the host for sleep monitoring device includes the host body, the top cover and the second magnet arranged on the inner surface of the top cover, the host body includes the housing and the first magnet arranged on the outer surface of the housing, and the host body and the top cover are pulled together by the first magnet and the second magnet, the bedspread is sandwiched between the host body and the top cover. Therefore, the problem regarding the fixation of the sleep monitoring device is solved, the fixation of the sleep monitoring device of the present disclosure is solid and the damage to the mattress and the sleep monitoring band is avoided.

### TECHNICAL FIELD

The present disclosure relates to medical device, particularly to a host for sleep monitoring device and sleep monitoring device thereof.

### BACKGROUND

When a sleep monitoring device is used on a bed, it is prone to slide down because of the gravity of its host body. In order to prevent the sleep monitoring device from sliding down, generally a tape is used to bind the sleep monitoring device onto the bed. However, in the case of binding the sleep monitoring device with a tape, not only the fixation is not solid, but also the mattress and the sleep monitoring band may be damaged, which will affect the aesthetic appearance.

### SUMMARY

The present disclosure provides a host for sleep monitoring device and the sleep monitoring device thereof.

According to the first aspect of the present disclosure, a host for sleep monitoring device is provided which may include a host body and a switch arranged on the host body. The host for sleep monitoring device may further include a top cover and a second magnet arranged on the inner surface of the top cover. The host body may include a housing and a first magnet arranged on the outer surface of the housing. The host body and the top cover may be pulled together by the first magnet and the second magnet.

In the host for sleep monitoring device mentioned above, there may be a plurality of first magnets which may be uniformly arranged at the periphery of the housing.

In the host for sleep monitoring device mentioned above, there are four first magnets which are symmetrically arranged with respect to a transverse centerline and a longitudinal centerline.

In the host for sleep monitoring device mentioned above, there are four second magnets which are symmetrically arranged with respect to a transverse centerline and a longitudinal centerline.

In the host for sleep monitoring device mentioned above, the switch may be a magnetic switch, a tact switch or a touch switch.

In the host for sleep monitoring device mentioned above, the magnetic switch may include a third magnet and a magnetic induction chip. The third magnet may be arranged on the inner surface of the top cover. The magnetic induction chip may be arranged on the host body. The third magnet cooperates with the magnetic induction chip.

The third magnet may be arranged below a transverse centerline of the top cover and the magnetic induction chip may be arranged below a transverse centerline of the host body.

In the host for sleep monitoring device mentioned above, the outer surface of the housing may be provided with an indictor, the top cover may be provided with a transparent cover, and a location of the transparent cover corresponds to that of the indictor.

In the host for sleep monitoring device mentioned above, the transparent cover is annular.

According to the second aspect of the present disclosure, a sleep monitoring device is provided which may include a sleep monitoring band and a host for sleep monitoring device mentioned above. One end of the sleep monitoring band is connected with the host for sleep monitoring device.

Because of the technical solutions mentioned above, the present disclosure has advantages as following:

(1) In the embodiments of present disclosure, since the host for sleep monitoring device includes the host body, the top cover and the second magnet arranged on the inner surface of the top cover, the host body includes the housing and the first magnet arranged on the outer surface of the housing, and the host body and the top cover are pulled together by the first magnet and the second magnet, the bedspread is sandwiched between the host body and the top cover when it is used. Therefore, the problem regarding the fixation of the sleep monitoring device is solved, the fixation of the sleep monitoring device of the present disclosure is solid and the damage to the mattress and the sleep monitoring band is avoided.

(2) In the embodiments of the present disclosure, the magnetic switch is used which includes the third magnet arranged on the inner surface of the top cover and the magnetic induction chip arranged on the host body. The third magnet cooperates with the magnetic induction chip. When the top cover is forwardly placed on the host body, the host body is powered-on and operates normally. When the top cover is rotated by 180 degree and then placed on the host body, a low level is output and the host body is powered off. The magnetic switch of the present disclosure has ingenious structures and is easy to use.

### BRIEF DESCRIPTION OF DRAWINGS

FIG. 1 is a schematic view showing the structure of a host body according to an embodiment of the present disclosure;

FIG. 2 is a schematic view showing the structure of a top cover according to an embodiment of the present disclosure;

FIG. 3 is a schematic view showing the structure of a sleep monitoring device according to an embodiment of the present disclosure; and

FIG. 4 is a schematic view showing the use state of the sleep monitoring device according to an embodiment of the present disclosure.

### DETAILED DESCRIPTION

The present disclosure will be described in details with reference to specific embodiments in connection with the drawings.

### The First Embodiment

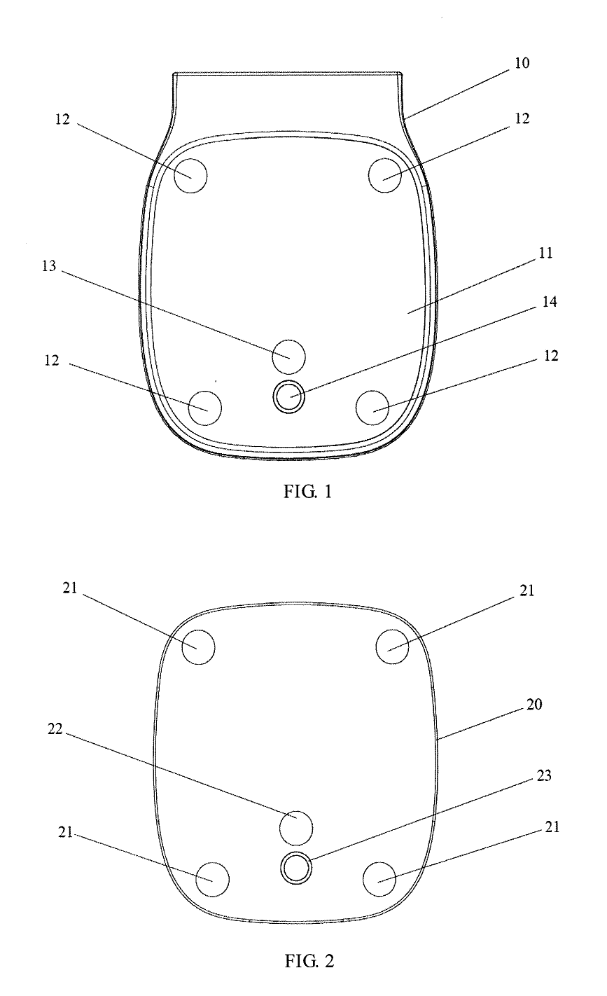

As shown in FIG. 1 and FIG. 2, according to an embodiment of the present disclosure, a host for sleep monitoring device may include a host body 10, a switch, a top cover 20 and second magnet 21. There may be one or more second magnets 21. The second magnet 21 may be arranged on the inner surface of the top cover 20. The host body 10 may include a housing 11 and first magnet 12. There may be one or more first magnets 12. The first magnet 12 may be arranged on the outer surface of the housing 11 and the location of the first magnet 12 may correspond to that of the second magnet 21. The switch may be arranged on the host body 10, specifically on the outer surface of the housing 11. The host body 10 and the top cover 20 are pulled together by the second magnet 21 and the first magnet 12.

There may be a plurality of first magnets 12 which may be uniformly arranged at the periphery of the housing 11, and the second magnets 21 may cooperate with the first magnets 12. In an embodiment, there are four first magnets 12 which are provided at four corners of the housing and symmetrically arranged with respect to the transverse centerline and the longitudinal centerline, respectively. Also, there are four second magnets 21 which are provided at four corners of the top cover 20 and symmetrically arranged with respect to the transverse centerline and the longitudinal centerline. There may also be two, three, five or six first magnets and second magnets, as long as they are symmetrically arranged such that the first magnets 12 will be respectively aligned with the second magnets 21 after the top cover 20 is rotated by 180 degree.

The switch may be a magnetic switch, a tact switch or a touch switch. In an embodiment, the switch is a magnetic switch which may include a third magnet 22 and a magnetic induction chip 13. The third magnet 22 may be arranged on the inner surface of the top cover 20 and the magnetic induction chip 13 may be arranged on the host body 10 and exposed on the surface. The third magnet 22 may cooperate with the magnetic induction chip 13. The magnetic induction chip 13 may be arranged at the lower part of the host body 10. The third magnet 22 may be a magnetic sheet and placed at the lower part of the top cover 20. The output terminal of the magnetic induction chip 13 may be connected to the enable terminal of the power chip of the host body. When the top cover 20 is forwardly placed on the host body 10, the magnetic induction chip 13 will sense the magnetic field of the magnetic sheet and output a high level, therefore the host body is powered-on and operates normally. When the top cover 20 is rotated by 180 degree and then placed on the host body 10, the third magnet 22 is misaligned with the magnetic induction chip 13 and there is no magnetic field around the magnetic induction chip 13, therefore a low level is output and the host body 10 is powered off.

The outer surface of the main housing 11 may be provided with an indicator 14 and the top cover 20 may be provided with a transparent cover 23. The location of the transparent cover 23 corresponds to that of the indicator 14 such that the indicator 14 can be observed through the transparent cover 23. The shape of the transparent cover 23 may be circle, square, oval, triangle, annular or other shape. In the present embodiment, the transparent cover 23 is annular.

### The Second Embodiment

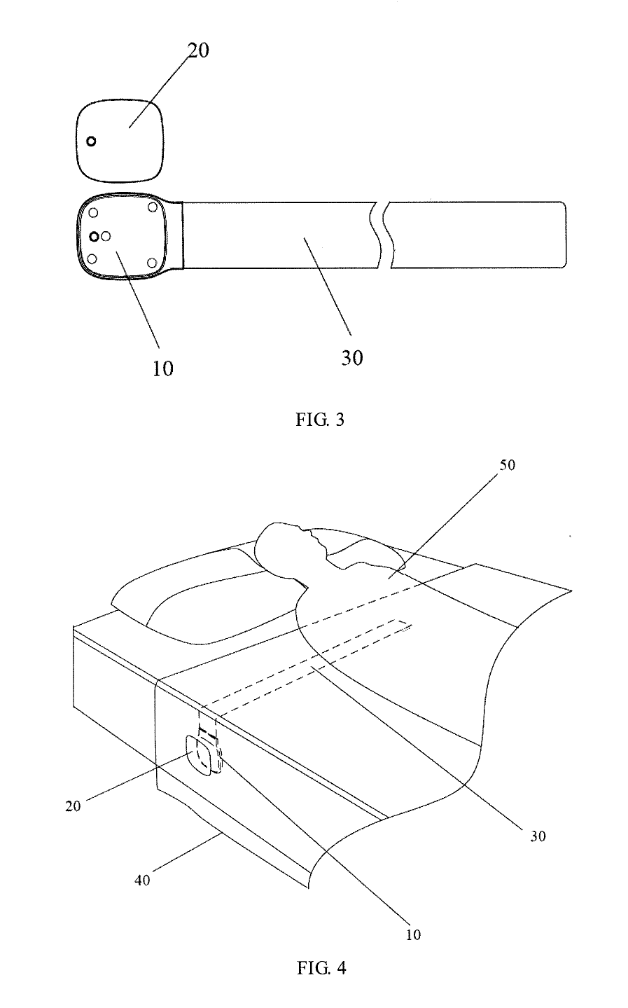

As shown in FIG. 1 to FIG. 3, according to an embodiment of the present disclosure, a sleep monitoring device may include a sleep monitoring band 30 and a host for sleep monitoring device. One end of the sleep monitoring band 30 is connected with the host for sleep monitoring device. The host for sleep monitoring device may include a host body 10, a switch, a top cover 20 and second magnet 21. There may be one or more second magnets 21. The second magnet 21 may be arranged on the inner surface of the top cover 20. The host body 10 may include a housing 11 and first magnet 12. There may be one or more first magnets 12. The first magnet 12 may be arranged on the outer surface of the housing 11 and the location of the first magnet 12 may correspond to that of the second magnet 21. The switch may be arranged on the host body 10, specifically on the outer surface of the housing 11. The host body 10 and the top cover 20 are pulled together by the second magnet 21 and the first magnet 12.

There may be a plurality of first magnets 12 which may be uniformly arranged at the periphery of the housing 11, and the second magnets 21 may cooperate with the first magnets 12. In an embodiment, there are four first magnets 12 which are provided at four corners of the housing and symmetrically arranged with respect to the transverse centerline and the longitudinal centerline, respectively. Also, there are four second magnets 21 which are provided at four corners of the top cover 20 and symmetrically arranged with respect to the transverse centerline and the longitudinal centerline. There may also be two, three, five or six first magnets and second magnets, as long as they are symmetrically arranged such that the first magnets 12 will be respectively aligned with the second magnets 21 after the top cover 20 is rotated by 180 degree.

The switch may be a magnetic switch, a tact switch or a touch switch. In an embodiment, the switch is a magnetic switch which may include a third magnet 22 and a magnetic induction chip 13. The third magnet 22 may be arranged on the inner surface of the top cover 20 and the magnetic induction chip 13 may be arranged on the host body 10 and exposed on the surface. The third magnet 22 may cooperate with the magnetic induction chip 13. The magnetic induction chip 13 may be arranged at the lower part of the host body 10. The third magnet 22 may be a magnetic sheet and placed at the lower part of the top cover 20. The output terminal of the magnetic induction chip 13 may be connected to the enable terminal of the power chip of the host body. When the top cover 20 is forwardly placed on the host body 10, the magnetic induction chip 13 will sense the magnetic field of the magnetic sheet and output a high level, therefore the host body is powered-on and operates normally. When the top cover 20 is rotated by 180 degree and then placed on the host body 10, the third magnet 22 is misaligned with the magnetic induction chip 13 and there is no magnetic field around the magnetic induction chip13, therefore a low level is output and the host body 10 is powered off.

The outer surface of the main housing 11 may be provided with an indicator 14 and the top cover 20 may be provided with a transparent cover 23. The location of the transparent cover 23 corresponds to that of the indicator 14 such that the indicator 14 can be observed through the transparent cover 23. The shape of the transparent cover 23 may be circle, square, oval, triangle, annular or other shape. In the present embodiment, the transparent cover 23 is annular.

FIG. 4 is a schematic view showing the sleep monitoring device in a use state, where the patient 50 is lying in bed, the sleep monitoring band 30 is arranged under the bedspread 40, the end of the sleep monitoring band 30 connected with the host body 10 and the host body 10 are suspended at one side of the bed, and the top cover 20 and the host body 10 are pulled together with the bedspread therebetween to achieve the fixation of the sleep monitoring device.

The foregoing is the further detail description of the present disclosure made with reference to the specific embodiments. However, it should not be interpreted as that the specific embodiments of the present disclosure are limited to the description. A number of simple deductions and alternatives may be made by person ordinary skilled in the art to which the present disclosure belong without departing from the concepts of the present disclosure.

We claim:

1. A host for sleep monitoring device, comprising a host body and a switch arranged on the host body, wherein the host for sleep monitoring device further comprises a top cover and a second magnet arranged on an inner surface of the top cover, the host body comprises a housing and a first magnet arranged on an outer surface of the housing, and the host body and the top cover are pulled together by the first magnet and the second magnet to fix the host onto a sheet which is sandwiched between the host body and the top cover.

2. The host for sleep monitoring device of claim 1, wherein there are a plurality of first magnets which are uniformly arranged at a periphery of the housing.

3. The host for sleep monitoring device of claim 2, wherein there are four first magnets which are symmetrically arranged with respect to a transverse centerline and a longitudinal centerline.

4. The host for sleep monitoring device of claim 3, wherein there are four second magnets which are symmetrically arranged with respect to a transverse centerline and a longitudinal centerline.

5. The host for sleep monitoring device of claim 1, wherein the switch is a magnetic switch, a tact switch or a touch switch.

6. The host for sleep monitoring device of claim 5, wherein the magnetic switch comprises a third magnet and a magnetic induction chip, the third magnet is arranged on the inner surface of the top cover, the magnetic induction chip is arranged on the host body, and the third magnet cooperates with the magnetic induction chip.

7. The host for sleep monitoring device of claim 6, wherein the third magnet is arranged below a transverse centerline of the top cover and the magnetic induction chip is arranged below a transverse centerline of the host body.

8. The host for sleep monitoring device of claim 1, wherein the outer surface of the housing is provided with an indictor, the top cover is provided with a transparent cover, and a location of the transparent cover corresponds to that of the indictor.

9. The host for sleep monitoring device of claim 8, wherein the transparent cover is annular.

10. A sleep monitoring device, comprising a sleep monitoring band and the host for sleeping monitoring device of claim 1, wherein one end of the sleep monitoring band is connected with the host for sleep monitoring device.
